SEQUENCE LISTING (27 sequences)

SEQ ID NO: 1 - DNA, 18 bp, Artificial Sequence
  Oligonucleotide
agtcgcatgacccaagac

SEQ ID NO: 2 - DNA, 18 bp, Artificial Sequence
  Oligonucleotide
caactgtgtgtatgtcac

SEQ ID NO: 3 - DNA, 18 bp, Artificial Sequence
  Oligonucleotide
gatggtgaaagggatttt

SEQ ID NO: 4 - DNA, 18 bp, Artificial Sequence
  Oligonucleotide
ggtcccatgatgcaagac

SEQ ID NO: 5 - DNA, 18 bp, Artificial Sequence
  Oligonucleotide
caactgtgtatatgtcac

SEQ ID NO: 6 - DNA, 18 bp, Artificial Sequence
  Oligonucleotide
caactgtgcatatgtcac

SEQ ID NO: 7 - DNA, 18 bp, Artificial Sequence
  Oligonucleotide
caactgtgcgtatgtcac

SEQ ID NO: 8 - DNA, 18 bp, Artificial Sequence
  Oligonucleotide
ggtcccatgatgcaagac

SEQ ID NO: 9 - DNA, 18 bp, Artificial Sequence
  Oligonucleotide
cccgtggtgatcatggtc

SEQ ID NO: 10 - DNA, 18 bp, Artificial Sequence
  Oligonucleotide
gtgacatacacacagttg

SEQ ID NO: 11 - DNA, 18 bp, Artificial Sequence
  Oligonucleotide
gtgacatacgcacagttg

SEQ ID NO: 12 - DNA, 18 bp, Artificial Sequence
  Oligonucleotide
gtgacgtacacacagttg

SEQ ID NO: 13 - DNA, 18 bp, Artificial Sequence
  Oligonucleotide
gtgacgtacgcacagttg

SEQ ID NO: 14 - DNA, 18 bp, Artificial Sequence
  Oligonucleotide
acctgactgtcgtgctcg

SEQ ID NO: 15 - DNA, 24 bp, Artificial Sequence
  Oligonucleotide
acctacagatgttatggttctgtt

SEQ ID NO: 16 - DNA, 21 bp, Artificial Sequence
  Oligonucleotide
tctacattaatacagaggcac

SEQ ID NO: 17 - DNA, 18 bp, Artificial Sequence
  Oligonucleotide
atctatagaaagcagact

SEQ ID NO: 18 - DNA, 18 bp, Artificial Sequence
  Oligonucleotide
attccctcacgtcattgt

SEQ ID NO: 19 - DNA, 21 bp, Artificial Sequence
  Oligonucleotide
agtaaacaaagaggaaccttc

SEQ ID NO: 20 - DNA, 21 bp, Artificial Sequence
  Oligonucleotide
agcaaagaggaccaggattta

SEQ ID NO: 21 - DNA, 21 bp, Artificial Sequence
  Oligonucleotide
cacagtcctttgcatgcagat

SEQ ID NO: 22 - DNA, 18 bp, Artificial Sequence
  Oligonucleotide
atccactacactgatgac

SEQ ID NO: 23 - DNA, 18 bp, Artificial Sequence
  Oligonucleotide
gtcgatggatggtgaaga

SEQ ID NO: 24 - DNA, 23 bp, Artificial Sequence
  Oligonucleotide
taccactggcatcgtgatggact

SEQ ID NO: 25 - DNA, 23 bp, Artificial Sequence
  Oligonucleotide
tccttctgcatcctgtcggcaat

SEQ ID NO: 26 - protein, 6 aa, Human
KIPFTI

SEQ ID NO: 27 - protein, 6 aa, Human
KLPFTI

(19) United States
(12) Patent Application Publication
(10) Pub. No.: US 2004/0259103 A1
(43) Pub. Date: Dec. 23, 2004

(54) Means of documenting repertoires of NKR immunoreceptors and/or of activatory or non-inhibitory immunoreceptor counterparts of NKR immunoreceptors

(76) Inventors: Eric Vivier, Cassis (FR); Frederic Vely, Cassis (FR)

Correspondence Address: BAKER & BOTTS, 30 ROCKEFELLER PLAZA, NEW YORK, NY, 10112

(21) Appl. No.: 10/633,771

(22) Filed: Aug. 4, 2003

Related U.S. Application Data
(63) Continuation of application No. 09/529,759, filed on Apr. 18, 2000.
Application No. 09/529,759 is a 371 of international application No. PCT/FR98/02244, filed on Oct. 20, 1998.

(30) Foreign Application Priority Data
Oct. 20, 1997 (FR) 97 13115

(51) Int. Cl. C12Q 1/68

(52) U.S. Cl. 435/6

(57) ABSTRACT

The invention relates to an in vitro method for documenting a repetoire in NKR immunoreceptors and/or NKR counterparts, consisting of (i) using at least a pair of oligonucleotides 3′ and 5′ capable of hybridizing with a target NKR receptor, or NKR counterpart, and not capable of being hybridized with a functional counterpart of said target receptor; (ii) contacting said pair of oligonucleotides 3′ and 5′ with the DNA or DNA of a sample under study; and (iii) detecting the ultimately formed hybrids. The invention also concerns the biological applications of said method, in particular for screening banks of organs, tissues and cells for transplant, and kits for its implementation.

The present invention relates to means which make it possible to document the repertoires, in an individual or an animal, of NKR (Natural Killer Receptor) immunoreceptors of the immunoglobulin type or of the lectin type, and of activatory, or at the very least non-inhibitory, immunoreceptor counterparts of NKR immunoreceptors. It also relates to their biological applications.

The immune functions in humans or animals are defined by several categories of highly diversified molecules, such as in particular the ABO blood group system, the family of MHC (Major Histocompatibility Complex, called, in humans, HLA—Human Leukocyte Antigen—system) molecules, the family of receptors for the T lymphocyte antigen (TCR) and B lymphocyte antigen (BCR). All the molecules which an adult individual expresses, or is capable of expressing, for each of these different families constitute, with the exception of identical twins, an evolutive repertoire which is specific to them and which is involved in self or non-self recognition.

Other main repertoires have been more recently identified. They are the immunoglobulin-type NKR receptor repertoire and the lectin-type NKR receptor repertoire. The immunoglobulin-type NKR receptors comprise the KIR receptors (Killer cell Inhibitory Receptors) such as in particular the p58.1, p58.2, p70.INH and p140.INH receptors. The lectin-type NKR receptors comprise the inhibitory NKG2 receptors such as in particular the NKG2A and NKG2B receptors. All these NKR receptors have an inhibitory function. Receptors which are highly homologous to them, in particular at the extracytoplasmic level, however fulfil activatory, or at the very least non-inhibitory, functions: they are the KAR receptors (Killer cell Activatory Receptors) which are homologous to the KIR receptors, and NKG2C, NKG2D, NKG2E and NKG2F receptors which are homologous to the NKG2A and NKG2B receptors. The activatory, or at the very least non-inhibitory, receptor counterparts of the inhibitory NKR receptors are hereinafter designated, for the sake of convenience, NKR counterparts.

The NKR receptors and the NKR receptor counterparts are naturally expressed by the NK cells and by subpopulations of T cells. Several of these receptors may be expressed by the same cell. All these receptors, whether they are inhibitory (i.e. NKR) or activatory or non-inhibitory (i.e. NKR counterparts), have in common the fact that they have as ligand molecules which are not antigen-derived: the ligands for the NKR receptors and for the NKR receptor counterparts are MHC class I molecules.

The recognition of its ligand by an NKR receptor triggers the transduction, to the cell, of a message intended to inhibit its activity, e.g. reduction or termination of cytolysis, of secretion of cytokines, whereas the recognition of its ligand by an NKR receptor counterpart induces therein an activatory, or at the very least non-inhibitory, message. The result of the NKR receptors and NKR receptor counterparts thus activated by their ligands is a signal, which is negative or positive overall, for activation of the NK and/or T cells expressing them.

The NKR receptors and their counterparts thus participate in the positive or negative control of the allogenic reactions of a given immune system with respect to what it then considers as non-self for example, cancer cells or infected cells, or alternatively allo- or xeno-genic graft or transplant cells.

The NKR receptors and their counterparts indeed participate in the reactions between host and graft during a grafting (or transplantation) of cells, tissue or organ which exhibit(s) a degree of antigenic incompatibility with the host. The involvement of NKR receptors and their counterparts in the tolerance towards incompatible grafts, and in the selective effect of lysis of malignant cells sometimes observed after a bone marrow graft, or GVL (Graft Versus Leukemia) effect, has indeed been demonstrated in vivo (cf. Cambiaggi et al. 1997, Proc. Natl. Acad. Sci. USA, vol. 94, p. 8088-8092; Albi et al. 1996, Blood, vol. 87, No. 9, p. 3993-4000).

However, the means which can currently be used in a medical context do not make it possible to document all the repertoires of a patient, of an organ, of a tissue or of cells.

Accordingly, only the compatibility of the HLA-A, HLA-B and HLA-DR molecules of the donor and of the recipient is currently checked prior to an allo- or xeno-genic graft or transplant. These compatibility criteria do not however appear to be satisfactory. Immunosuppressive treatments (for example based on cyclosporin) should supplement these graft or transplant procedures so as to inhibit the patient's immune system. Such treatments have high risks for the patient who is then likely to develop opportunistic infections. These immunosuppressive treatments must furthermore be maintained at a certain level for several years, and the patient then has to withstand the damaging effects of the medicaments. Finally, the success of such graft or transplant procedures remains uncertain. Indeed, graft rejections on the part of the recipient, or alternatively, in the case of grafts comprising immunocompetent cells, graft-versus-host reactions (GVH effect) are nevertheless observed. Such rejection or GVH reactions generally lead to very severe lesions; currently, they cannot however be completely avoided, and therefore prevented.

Unexpected beneficial effects of allogenic grafts have, moreover, sometimes been observed: allogenic grafts of bone marrow in aplastic leukaemia patients have sometimes led to a therapeutic antitumour effect through lysis of the malignant cells of the recipient and preservation of their healthy cells. This selective therapeutic effect, in which the NK and T cells are involved, is designated GVL (Graft Versus Leukemia). Potentially, a grafting (or a transplantation) of haematopoietic tissue in general, and of bone marrow in particular, can lead to a therapeutic effect in the context of haematological malignancies such as a leukaemia, through selective lysis of those cells from the recipient which no longer have the histocompatibility antigens presented by healthy cells. The means currently available to the medical setting do not make it possible, however, to predict if the organ or the tissue considered will exert a selective GVL effect for the recipient considered. Although known, the selective GVL effect cannot therefore be currently exploited in the context of an anticancer therapy.

The means currently developed in the context of experimental research studies in order to document the different repertoires of human or animal cells in fact do not make it possible to precisely document the repertoire of NKR receptors and of NKR receptor counterparts: the precise identity of each NKR receptor or NKR counterpart cannot be determined. Because of the strong homology, in particular at the extracytoplasmic level, between an NKR receptor and an NKR receptor counterpart (e.g. up to 96% homology between KIR and KAR), the use of antibodies indeed often do not make it possible to discriminate between NKRs which are inhibitory and their counterparts with activatory, or at the very least non-inhibitory, functions. The oligonucleotide primers which are currently available, for their part, do not allow the use of a polymerase chain reaction capable of discriminating between, for example, an NKR p58.1 and an NKR p58.2, or between an NKR p70.INH and an NKR p140.INH. Finally, to accurately document the repertoire of NKR immunoreceptors and of their counterparts, use must currently be made, after a step of purifying the desired receptors (e.g. by FACScan), of a nucleotide sequencing step. The documenting of the NKR/NKR counterpart repertoire cannot therefore be currently performed routinely in a medical-type context. The level of stimulation and inhibition of the programmes of activation of the NK and T cells, and therefore the potential of an individual to be resistant to the development of microbial or parasitic infections, of autoimmune diseases, or alternatively of malignant cells, cannot therefore be measured. The result of this lack of suitable means for documenting NKR/NKR counterpart repertoires is also that the selective GVL-type effects cannot be used in therapy, and that the GVH or rejection reactions during allo- or xeno-genic grafts or transplants cannot be completely avoided.

The present invention therefore provides means which make it possible to document, for a given biological sample, the repertoires of NKR immunoreceptors and of activatory or at the very least non-inhibitory immunoreceptor counterparts of NKR receptors. These means make it possible in particular to easily distinguish between an NKR receptor and its activatory or non-inhibitory counterpart, as well as to distinguish between various NKR receptors, or between various NKR counterparts. It also relates to the biological, and in particular medical and veterinary, applications of these means. One of the essential aspects according to the invention consists in considering all the NKR immunoreceptors and NKR counterparts as a repertoire, that is to say as a coherent set, forming a unit with respect to a type of activity, in this case, in a particularly advantageous manner, the control of lymphocyte activation in humans or animals from which the said biological sample is derived (negative control for the NKR receptor repertoire, positive control for the NKR counterpart receptor). The invention provides, for the first time, means which make it possible to document, routinely in a medical or veterinary context, NKR and/or NKR counterpart repertoires, so as to be able to rapidly and effectively analyse physiological and pathological situations linked to these repertoires.

The means according to the invention have in particular the advantage of being easy to use in a medical or veterinary context, for example in a hospital or clinic.

The subject of the present invention is an in vitro method of documenting a repertoire of (an) NKR immunoreceptor(s) comprising in particular the KIR p58.1, p58.2, p70.INH and p140.INH, and the NKG2A and NKG2B receptors, and/or of a repertoire of (an) NKR immunoreceptor(s) counterpart(s), comprising in particular the KAR p50.1, p50.2, p70.ACT and p140.ACT receptors, and the NKG2C, NKG2D, NKG2E and NKG2F receptors, these immunoreceptors being designated hereinafter target receptor(s), characterized in that it comprises: i. the use of at least one pair of oligonucleotides, one being designated 3′ oligonucleotide and the other 5′ oligonucleotide, the 3′ and 5′ oligonucleotides of the same said pair both being capable, under hybridization conditions corresponding to incubation for 1 min in a buffer [20 mM Tris-HC1, pH 8.4; 50 mM KCl; 2.5 mM MgCl₂] at a temperature of between 50° C. and 65° C. approximately, of hybridizing to the DNA or to the cDNA of a target NKR receptor, or NKR counterpart, but not hybridizing, under the same hybridization conditions, with the DNA or the cDNA of an NKR receptor counterpart, or respectively of an NKR receptor, functional counterpart of the said target receptor, ii. the bringing of DNA or cDNA populations of a biological sample of human or animal origin for which it is desired to document the repertoire of target immunoreceptor(s), into contact with an excess of at least one 3′ and 5′ oligonucleotide pair according to i. under conditions favourable to the hybridization of this 3′ and 5′ oligonucleotide pair with the DNAs or cDNAs of the biological sample, and iii. the detection of the possible hybrids formed between these DNAs or cDNAs and the 3′ and 5′ oligonucleotide pair(s).

Functional counterpart of a receptor is understood to mean in the present application a receptor with a homologous structure, in particular at the extracytoplasmic level, but with a different function: for example, a functional counterpart of the NKR p58.1 receptor is the NKR p50.1 receptor counterpart, and conversely; likewise the NKR p58.2 receptor and the NKR p50.2 receptor counterpart are functional counterparts for one another.

The present method therefore makes it possible in particular to distinguish an NKR receptor (or NKR counterpart) from a functional receptor counterpart of this receptor.

The 3′ and 5′ oligonucleotide pair(s) is (are) in particular capable of delimiting, on the DNA or the cDNA of a target receptor corresponding thereto, an oligonucleotide sequence (limits included) which is absent from the DNA or cDNA sequence of a receptor with which it (they) is (are) capable of not hybridizing under the hybridization conditions given under i) above.

Advantageously, the said or at least one of the said 3′ and 5′ oligonucleotide pair(s) used is in addition capable, under the same hybridization conditions as those defined under i., of not hybridizing to the DNA or cDNA of a receptor, either NKR or NKR counterpart, other than the said target receptor.

According to a particular arrangement of this advantageous manner, the said (or at least one of the said) 3′ and 5′ oligonucleotide pair(s) capable, under the hybridization conditions defined under i. above, of hybridizing to the DNA or the cDNA of a p58.1 (or p50.1) receptor, and of not hybridizing to the DNA or the cDNA of a p50.1 receptor (or respectively p58.1) is in addition capable of not hybridizing under the same hybridization conditions to the DNA or the cDNA of a p58.2 or p50.2 receptor.

According to a second particular arrangement of this advantageous manner, the said, or at least one of the said, 3′ and 5′ oligonucleotide pair(s) capable, under the hybridization conditions defined under i. above, of hybridizing to the DNA or the cDNA of a p58.2 (or p50.2) receptor, and of not hybridizing to the DNA or the cDNA of a p50.2 (or respectively p58.2) receptor is in addition capable of not hybridizing under the same hybridization conditions to the DNA or the cDNA of a p58.1 or p50.1 receptor.

According to a third particular arrangement of this advantageous manner, the said (or at least one of the said) 3′ and 5′ oligonucleotide pair(s) capable, under the hybridization conditions defined under i. above, of hybridizing to the DNA or the CDNA of a p70.INH (or p70.ACT) receptor, and of not hybridizing to the DNA or the cDNA of a p70.ACT (or respectively p70.INH) receptor is in addition capable of not hybridizing under the same hybridization conditions to the DNA or the cDNA of a p140.INH or p140.ACT receptor.

According to a fourth particular arrangement of this advantageous manner, the said, or at least one of the said, 3′ and 5′ oligonucleotide pair(s) capable, under the hybridization conditions defined under i. above, of hybridizing to the DNA or the cDNA of a p140.INH (or p140.ACT) receptor, and of not hybridizing to the DNA or the cDNA of a p140.ACT (or respectively p140.INH) receptor is in addition capable of not hybridizing under the same hybridization conditions to the DNA or the cDNA of a p70.INH or p70.ACT receptor.

According to an advantageous embodiment of the invention, the 5′ oligonucleotide of a said 3′ and 5′ oligonucleotide pair used for an NKR target receptor (or NKR counterpart) is capable, under the hybridization conditions defined under i. above, of hybridizing to the DNA or to the CDNA of an NKR receptor counterpart (or respectively NKR receptor), functional counterpart of the said NKR target receptor (or respectively NKR receptor counterpart) . According to an advantageous arrangement of this embodiment, the 5′ oligonucleotide sequence of a said 3′ and 5′ oligonucleotide pair used for an NKR target (or NKR counterpart) receptor comprises the 5′ oligonucleotide sequence of another said 3′ and 5′ oligonucleotide pair having as target receptor an NKR receptor counterpart (or respectively NKR receptor), functional counterpart of the said NKR target receptor (or respectively NKR receptor counterpart).

According to another advantageous embodiment of the invention, the 3′ oligonucleotide of a said 3′ and 5′ oligonucleotide pair, used for a KAR target receptor, is capable, under the same said hybridization conditions, of hybridizing to the DNA or cDNA of the said KAR target receptor at the level of a nucleotide stretch which comprises a sequence corresponding, according to the universal genetic code, and taking into account the degeneracy of the said code, to the amino acid sequence Lys Ile Pro Phe Thr Ile (KIPFTI) or Lys Leu Pro Phe Thr Ile (KLPFTI) (SEQ ID No. 26 or 27).

According to a particularly advantageous embodiment of the invention, the said (or at least one of the said) 3′ and 5′ oligonucleotide pair(s) having as target receptor a KIR receptor is chosen from the group of 3′ and 5′ oligonucleotide pairs consisting of: a 5′ oligonucleotide comprising the sequence SEQ ID No. 1, or a sequence which is derived therefrom, and at least one 3′ oligonucleotide comprising the sequence SEQ ID No. 5, No. 2, No. 6 or No. 7, or a sequence which is derived therefrom, a 5′ oligonucleotide comprising the sequence SEQ ID No. 4, or a sequence which is derived therefrom, and at least one 3′ oligonucleotide comprising the sequence SEQ ID No. 5, No. 2, No. 6 or No. 7, or a sequence which is derived therefrom, a 5′ oligonucleotide comprising the sequence SEQ ID No. 9, or a sequence which is derived therefrom, and at least one 3′ oligonucleotide comprising the sequence SEQ ID No. 5, No. 2, No. 6 or No. 7, or a sequence which is derived therefrom, at least one 5′ oligonucleotide comprising the sequence SEQ ID No. 10, No. 11, No. 12 or No. 13, or a sequence which is derived therefrom, and a 3′ oligonucleotide comprising the sequence SEQ ID No. 14, or a sequence which is derived therefrom.

Sequence which is derived from a first sequence is understood to mean in the present application a sequence derived from the first in particular by inversion, deletion, addition or substitution of nucleotide(s), and exhibiting the hybridization properties which the nucleic acid corresponding to the first sequence exhibits under the conditions i. defined above.

According to one arrangement of this particularly advantageous embodiment, the said 3′ and 5′ oligonucleotide pair having as target receptor a p58.1 receptor corresponds to a 5′ oligonucleotide comprising SEQ ID No. 1, or a sequence which is derived therefrom, and an equal mixture of four 3′ oligonucleotides, each of them comprising SEQ ID No. 5, No. 2, No. 6 or No. 7, or a sequence which is derived therefrom.

According to another arrangement of this particularly advantageous embodiment, the said 3′ and 5′ oligonucleotide pair having as target receptor a p58.2 receptor corresponds to a 5′ oligonucleotide comprising SEQ ID No. 4, or a sequence which is derived therefrom, and an equal mixture of four 3′ oligonucleotides, each of them comprising SEQ ID No. 5, No. 2, No. 6 or No. 7, or a sequence which is derived therefrom.

According to yet another arrangement of this particularly advantageous embodiment, the said 3′ and 5′ oligonucleotide pair having as target receptor a p70.INH receptor corresponds to a 5′ oligonucleotide comprising SEQ ID No. 9, or a sequence which is derived therefrom, and an equal mixture of four 3′ oligonucleotides, each of them comprising SEQ ID No. 5, No. 2, No. 6 or No. 7, or a sequence which is derived therefrom.

According to yet another arrangement of this particularly advantageous embodiment, the said 3′ and 5′ oligonucleotide pair having as target receptor a p140.INH receptor corresponds to an equal mixture of four 5′ oligonucleotides, each of them comprising SEQ ID No. 10, No. 11, No. 12 or No. 13, or a sequence which is derived therefrom, and a 3′ oligonucleotide comprising SEQ ID No. 14, or a sequence which is derived therefrom.

According to another particularly advantageous embodiment of the invention, the said (or at least one of the said) 3′ and 5′ oligonucleotide pair(s) having as target receptor a KAR receptor is chosen from the group of 3′ and 5′ oligonucleotide pairs consisting of:

a 5′ oligonucleotide comprising the sequence SEQ ID No. 1, or a sequence which is derived therefrom, and a 3′ oligonucleotide comprising the sequence SEQ ID No. 3, or a sequence which is derived therefrom,

a 5′ oligonucleotide comprising the sequence SEQ ID No. 8, or a sequence which is derived therefrom, and a 3′ oligonucleotide comprising the sequence SEQ ID No. 3, or a sequence which is derived therefrom,

a 5′ oligonucleotide comprising the sequence SEQ ID No. 9, or a sequence which is derived therefrom, and a 3′ oligonucleotide comprising the sequence SEQ ID No. 3, or a sequence which is derived therefrom,

a 5′ oligonucleotide comprising the sequence SEQ ID No. 15, or a sequence which is derived therefrom, and a 3′ oligonucleotide comprising the sequence SEQ ID No. 3, or a sequence which is derived therefrom.

According to one arrangement of this other particularly advantageous embodiment, the said 3′ and 5′ oligonucleotide pair having as target receptor a p50.1 receptor corresponds to a 5′ oligonucleotide comprising SEQ ID No. 1, or a sequence which is derived therefrom, and a 3′ oligonucleotide comprising SEQ ID No. 3, or a sequence which is derived therefrom.

According to another arrangement of this other particularly advantageous embodiment, the said 3′ and 5′ oligonucleotide pair having as target receptor a p50.2 receptor corresponds to a 5′ oligonucleotide comprising SEQ ID No. 8, or a sequence which is derived therefrom, and a 3′ oligonucleotide comprising SEQ ID No. 3, or a sequence which is derived therefrom.

According to yet another arrangement of this other particularly advantageous embodiment, the said 3′ and 5′ oligonucleotide pair having as target receptor a p70.ACT receptor corresponds to a 5′ oligonucleotide comprising SEQ ID No. 9, or a sequence which is derived therefrom, and a 3′ oligonucleotide comprising SEQ ID No. 3, or a sequence which is derived therefrom.

According to yet another arrangement of this other particularly advantageous embodiment, the said 3′ and 5′ oligonucleotide pair having as target receptor a p140.ACT receptor corresponds to a 5′ oligonucleotide comprising SEQ ID No. 15, or a sequence which is derived therefrom, and a 3′ oligonucleotide comprising SEQ ID No. 3, or a sequence which is derived therefrom.

According to yet another particularly advantageous embodiment of the invention, the said (or at least one of the said) 3′ and 5′ oligonucleotide pair(s) having as target receptor a NKG2 receptor is chosen from the group of 3′ and 5′ oligonucleotide pairs consisting of:

a 5′ oligonucleotide comprising the sequence SEQ ID No. 16, or a sequence which is derived therefrom, and a 3′ oligonucleotide comprising the sequence SEQ ID No. 17, or a sequence which is derived therefrom,

a 5′ oligonucleotide comprising the sequence SEQ ID No. 18, or a sequence which is derived therefrom, and a 3′ oligonucleotide comprising the sequence SEQ ID No. 17, or a sequence which is derived therefrom,

a 5′ oligonucleotide comprising the sequence SEQ ID No. 19, or a sequence which is derived therefrom, and a 3′ oligonucleotide comprising the sequence SEQ ID No. 17, or a sequence which is derived therefrom,

a 5′ oligonucleotide comprising the sequence SEQ ID No. 20, or a sequence which is derived therefrom, and a 3′ oligonucleotide comprising the sequence SEQ ID No. 21, or a sequence which is derived therefrom.

According to a first arrangement of this yet another particularly advantageous embodiment, the said 3′ and 5′ oligonucleotide pair having as target receptor an NKG2A (inhibitor) receptor corresponds to a 5′ oligonucleotide comprising SEQ ID No. 16, or a sequence which is derived therefrom, and a 3′ oligonucleotide comprising SEQ ID No. 17, or a sequence which is derived therefrom.

According to a second arrangement of this yet another particularly advantageous embodiment, the said 3′ and 5′ oligonucleotide pair having as target receptor an NKG2B (inhibitor) receptor corresponds to a 5′ oligonucleotide comprising SEQ ID No. 18, or a sequence which is derived therefrom, and a 3′ oligonucleotide comprising SEQ ID No. 17, or a sequence which is derived therefrom.

According to a third arrangement of this yet another particularly advantageous embodiment, the said 3′ and. 5′ oligonucleotide pair having as target receptor an NKG2C (activator) receptor corresponds to a 5′ oligonucleotide comprising SEQ ID No. 19, or a sequence which is derived therefrom, and a 3′ oligonucleotide comprising SEQ ID No. 17, or a sequence which is derived therefrom.

According to a fourth arrangement of this yet another particularly advantageous embodiment, the said 3′ and 5′ oligonucleotide pair having as target receptor an NKG2D (activator) receptor corresponds to a 5′ oligonucleotide comprising SEQ ID No. 20, or a sequence which is derived therefrom, and a 3′ oligonucleotide comprising SEQ ID No. 21, or a sequence which is derived therefrom.

The said conditions which are favourable for the hybridization of the 3′ and 5′ oligonucleotide pair(s) brought into contact with the DNA or the cDNA of the biological sample advantageously correspond to an incubation for 1 min in a buffer [20 mM Tris-HCl, pH 8.4; 50 mM KCl; 2.5 mM MgCl₂] at a temperature of between 50° C. and 65° C. approximately. Such conditions are in particular presented in the examples.

Advantageously, the two 3′ or 5′ oligonucleotides of the same said pair are each coupled to a marker, in particular coupled to a fluorescent or radioactive marker, such as ³²p allowing the visualization of the hybrids which they may form with the said DNA or cDNA populations of the said biological sample.

In an equally advantageous manner, the said 3′ and 5′ oligonucleotide pair(s) serve(s) as 3′ and 5′ primers, respectively, for extension by DNA polymerase, such as a Taq polymerase. Conditions which are favourable for such an extension comprise, apart from the addition of DNA polymerase, the addition of the 4 dNTPs (deoxyribonucleoside triphosphates) in the presence of a Tris-HCl-type buffer.

The said hybrids which may be formed are then, prior to their detection, amplified by at least one PCR (amplification by the polymerase chain reaction; cf. patents EP 201,184 and EP 200,362) or RT-PCR in the case of cDNA retrotranscribed from MRNA. Where appropriate, the said hybrids which may be formed are amplified by nested PCR. Examples of conditions which are favourable for the PCR amplification are given in the examples.

According to another advantageous embodiment of the invention, the said detection of the hybrids which may be formed comprises, in addition, the resolution, on a polyacrylamide gel, of, the reaction mixture derived from the bringing into contact, as well as the visualization of the presence or of the absence of electrophoretic bands containing the said hybrids which may be formed.

According to another embodiment of the invention, the documented immunoreceptor repertoire is quantified with reference to the quantities of β-actin measured in the same biological sample, or with reference to the quantities of a specific molecule of a cellular type which are present in the said biological sample, such as in particular the CD56 molecules for the NK cells.

The method according to the invention may be applied to the documentation of a genotypic repertoire of NKR immunoreceptors and/or of NKR immunoreceptor counterparts: step ii. of the bringing into contact defined above is then carried out with the genomic DNA populations of the biological sample.

The method according to the invention may also be applied to the documentation of an expression repertoire of NKR immunoreceptors and/or of NKR immunoreceptor counterparts: step ii. of the bringing into contact defined above is then carried out with the cDNA populations retrotranscribed from the mRNA populations of the biological sample.

Biological samples of human or animal origin which are particularly appropriate for carrying out the method according to the invention comprise peripheral blood, bone marrow, lymphocytes, NK and/or T cells, transgenic cells expressing immunoreceptors and a fraction isolated from these samples.

The method according to the invention may be applied in particular to the screening of a library of organs, tissues or cells.

It thus allows better prediction:

of the acceptance or rejection, by a human or an animal, of cells, of a tissue or of an organ which is (are) genetically different,

of the safety or pathogenicity (GVH effect), for a human or an animal, of a graft or transplant, in particular of cells, tissue or organ which is (are) genetically different,

of a potential effect of the GVL type which cells, a tissue or an organ which is (are) genetically different could exert on a human or an animal.

The method according to the invention also allows the monitoring of the possible appearance of such reactions after allo- or xeno-genic grafting or transplantation.

The method according to the invention can also be applied to the determination of the state of activation of NK and/or T cells at a given instant in an animal or a human. It allows, in this case, the prediction or monitoring of the state of resistance of an animal or a human towards a viral infection, such as an HIV infection, or a parasitic infection, such as malaria, or a bacterial infection, towards an autoimmune disease, such as rheumatoid arthritis, or alternatively towards the development of malignant cells such as leukaemia cells. The predictive use of the method according to the invention is of particular importance in the context of epidemics.

The method according to the invention can also be advantageously applied to the screening of medicaments which are active on infectious diseases, on autoimmune diseases and on tumour diseases.

The subject of the present invention is also a kit for carrying out the said method comprising, in a container, at least one said oligonucleotide pair, the reagents for carrying out the said method(s) such as a buffer, a marker (optionally coupled to the oligonucleotides of the said pair), as well as instructions for use.

Other characteristics and advantages of the present invention will further emerge from the following exemplary embodiments which are given as a guide and without limitation.

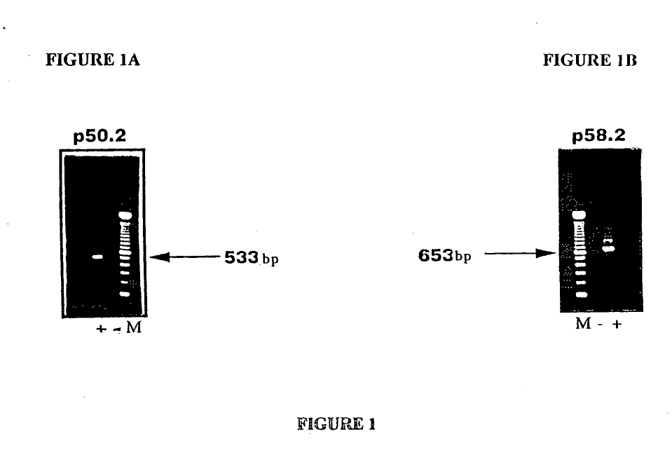

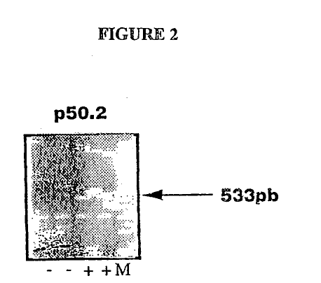

The said examples refer to FIGS. 1 and 2:

FIG. 1 represents the products derived from a PCR amplification (amplification by the polymerase chain reaction after enzymatic reverse transcription, RT, with the aid of oligonucleotide pairs according to the invention serving as primers), of the sequences encoding p50.2 (FIG. 1A) and p58.2 (FIG. 1B) in human NK cells;

FIG. 2 represents the products derived from a PCR amplification of the sequence encoding p50.2 from the genomic DNA of p50.2⁺ transgenic mice.

### EXAMPLE 1

Documentation of the NKR/NKR Counterpart Repertoire Expressed by a Population of Human NK Cells (RT-PCR).

1. Preparation of the RNAs

RNA preparations were made from cloned human NK cells phenotyped p50.2⁺ and/or p58.2⁺. Immunological technique does not make it possible to accurately document such a repertoire: the antibody GL183 (Immunotech) recognizes both the inhibitory NKR receptor p58.2 and its activatory counterpart p50.2. Cloned human NK cells phenotyped p50.2⁻ and p58.2⁻ with the aid of the antibody. GL183 serve as negative controls.

The RNA preparations are made as follows.

Extraction

100 μl of Trizol (Gibco BRL category No. 15596-026) were added to 10⁶ cells. The medium is mixed by pipetting several times, without using a vortex mixer. The solution is left for 5 minutes at room temperature and then 20 μl of chloroform, free of isoamyl alcohol, are added. The medium is again mixed without using a vortex mixer and the solution is allowed to stand for 5 minutes at room temperature. It is then centrifuged at 4° C. for 15 minutes so as to properly separate the bottom organic phase, which contains the DNA, from the top aqueous phase which contains the RNA. The aqueous phase is recovered without disturbing the interface between the aqueous phase and the organic phase.

Precipitation

50 μl of isopropanol are added to the aqueous phase and the RNA is allowed to precipitate for 15 minutes at room temperature. The medium is then centrifuged for 10 minutes at 4° C. The supernatant is removed, and the pellet is washed with 100 μl of 70% ethanol. After centrifuging for 5 minutes at 4° C. (7500 g), the medium is allowed to dry in the open air (without drying under vacuum). The RNA pellet is resuspended in 20 ml of H₂O.

2. Preparation of the Oligonucleotide Pairs

Table 1 below presents the oligonucleotide pairs used. Reported here are the results relating to the use of the oligonucleotide pairs C (SEQ-ID No. 4 as 5′ oligonucleotide and an equal mixture of SEQ ID No. 5, No. 2, No. 6 and No. 7 as 3′ oligonucleotide) and D (SEQ ID No. 8 as 5′ oligonucleotide and SEQ ID No. 3 as 3′ oligonucleotide) which are presented in Table 1. The cDNA sequences, on, the basis of which these oligonucleotide pairs were developed, are presented in Table 2 below (name of the cDNA clones and Genbank accession number). For each oligonucleotide pair, the allelic variants and the excision-splicing variants (alternative splicing) known for the same receptor were thus taken into account.

Each oligonucleotide pair is constructed, after alignment of the known cDNA sequences of the different variants of the same target receptor (e.g. KIR p58.2), so that this pair can determine, on all these variants, the limits of a consensus fragment, without being able, as a result, to do likewise on any variant of the receptor counterpart of the target receptor (e.g. KAR p50.2). The sequence of each oligonucleotide of the same pair is then optimized so that the annealing temperature for each of them is similar (e.g. ΔT≦5° C.).

Each oligonucleotide indeed has an annealing temperature which is specific to it. This annealing temperature depends on the ratio

$$R = \frac{G + C}{total number of bases} \times 100 and on the$$

length of the oligonucleotide considered, according to the formula: annealing temperature of an oligonucleotide=

$$Tm = 69.3 + 0.41 ( R ) - \frac{650}{length in bp} ( {in }^{°} C . )$$

However, in a reaction of the polymerase chain reaction type, the oligonucleotides of the same pair should both be able to anneal to the target receptor under common reaction conditions, this being so as to serve as primers for the amplification of the consensus fragment. If the oligonucleotides of the same pair have similar specific annealing temperatures (e.g. 54° C. and 56° C.), they will be able to hybridize to the target receptor, without, as a result, hybridizing to the corresponding receptor counterpart, at a temperature of 54° C. or 55° C.

If the oligonucleotides of the same pair have, on the other hand, very different specific annealing temperatures (e.g. 49° C. and 56° C.), the reaction for hybridizing to the target receptor is preferably carried out at the lower of the two temperatures (e.g. 49° C. or 50° C.), which makes it possible to maintain the recognition of its nucleotide target by the oligonucleotide whose specific annealing temperature is the lowest. In this situation, a decrease in specificity can however occur: it is possible to observe that some oligonucleotide pairs succeed, under such temperature conditions, in hybridizing to the target receptor counterpart-receptor. A way of counteracting this loss of specificity consists in increasing the length of the oligonucleotide whose specific annealing temperature is the lowest, without causing the oligonucleotide pair considered to lose its specificity.

3. Amplification by the Polymerase Chain Reaction After Enzymatic Reverse Transcription (RT-PCR)

5 μg of total RNA are transcribed into cDNA by incubating with a reverse transcriptase (RT) with the aid of the First Strand DNA-Ready to go kit (Pharmacia). 10 μl of cDNA out of the 33 μl obtained are brought into contact with oligonucleotide pairs C and D which serve, in this case, as primers (cf. Table 1): 10 μl of RT-derived product; 10X PCR buffer: 10 μl; MgCl₂ 50 mM; dXTP 10 mM; 3′ oligonucleotide at 10 μM: 5 μl; 5′ oligonucleotide 10 μM: 5 μl; Taq polymerase: 0.5 l; H₂O: qs 100 μl. The PCR amplification (DNA engine PTC 200, MJ Research, Massachusetts) is carried out according to the following steps:

step No. 1 (initial denaturation): 5 min at 94° C.,

step No. 2: 35 cycles comprising

a) denaturation 1 min at 94° C.

b) annealing 1 min at 55° C. for the oligonucleotide pair C and 50° C. for the oligonucleotide pair D,

c) extension 1 min at 72° C.,

step No. 3: (final extension):

1 min at 72° C.

The duration of extension 2c can be increased if the fragment to be amplified is of a large size (e.g. greater than 1000-1400 bp approximately).

The temperature in the annealing step 2b depends on the pair of oligonucleotides used as primers (cf. point 2. above). It corresponds to a consensus temperature between the annealing temperatures specific for each of the two oligonucleotides forming a pair (mean temperature or the lower of the two temperatures). This temperature is generally between 45° C. and 70° C., preferably between 50° C. and 65° C.

10 μl of the praducts derived from the amplification by RT-PCR are resolved by electrophoresis on a 2% agarose gel in parallel with molecular weight markers (M).

The results are illustrated in FIGS. 1A and 1B.

FIG. 1 illustrates the PCR amplification after RT (enzymatic reverse transcription) of the sequences encoding p58.2 and p50.2 of human NK cells.

In FIG. 1A is illustrated the result of the electrophoretic resolution of the products derived from the RT-PCR amplification after bringing the pair of primers D according to the invention (cf. Table 1) into contact with the cDNA populations of human NK cells phenotyped p50.2⁺ and p58.2⁺ (lane+) with the aid of the antibody GL183, or with the cDNA populations of human NK cells phenotyped p50.2⁻ and p58.2⁻ (lane−) with the aid of this same antibody GL183. The molecular weight markers are resolved in lane M.

In FIG. 1B is illustrated the result of the electrophoretic resolution of the products derived from the RT-PCR amplification after bringing the pair of primers C according to the invention (cf. Table 1) into contact with the cDNA populations of human NK cells phenotyped p50.2⁺ and p58.2⁺ (lane+) with the aid of the antibody GL183, or with the cDNA populations of human NK cells phenotyped p50.2⁻ and p58.2⁻ (lane−) with the aid of this same antibody GL183. The molecular weight markers are resolved in lane M.

It can be observed that the pairs of oligonucleotides C and D according to the invention make it possible to recognize respectively a phenotype, respectively, p58.2³⁰ and p50.2⁺, by recognizing a fragment of, respectively, 653 bp and 533 bp. The method according to the invention therefore makes it possible to discriminate between a p58.2⁺ phenotype (KIR receptor, with inhibitory function) and a p50.2⁺ phenotype (KAR receptor counterpart of p58.2, with activatory function), which up until now could not be carried out by sequencing.

### EXAMPLE 2

Documentation of the NKR/NKR Counterpart (Potential) Genetic Repertoire of a Population of p50.2⁺ transgenic mouse splenocytes (PCR).

1—Preparation of the DNAs

DNA preparations were carried out using p50.2⁺ transgenic mouse splenocytes. Immunological technique does not make it possible to determine if such splenocytes are p50.2⁺ (KAR receptor, activatory) or p58.2⁺ (KIR receptor, inhibitory, or alternatively p50.2⁺ and p58.2⁺) . Non-transgenic mouse splenocytes (p50.2⁻) serve as negative controls.

Extraction

This step is carried out as described in Example 1. The DNAs being contained in the bottom organic phase, it is this phase which is recovered here after having removed the aqueous phase and a small amount of interface.

Precipitation

30 μl of 100% ethanol are added and the medium is allowed to stand for 5 minutes at room temperature. After centrifuging for 5 minutes at 4° C. (2000 g), the supernatant is discarded and the pellet is washed with 100 μl of 0.1 M sodium citrate in 10% ethanol. The medium is left for 30 minutes at room temperature while mixing from time to time. It is centrifuged for 5 minutes at 4° C. (2000 g) . This washing is repeated a second time.

The DNA pellet obtained is resuspended in 200 μl of 70% ethanol. The medium is left for 15 minutes at room temperature while mixing from time to time and centrifuged for 5 minutes at 4° C. (2000 g).

The pellet is left to dry briefly under vacuum (1 to 2 μg of DNA approximately are obtained) and is resuspended in 10 μl of 8 mM NaOH. If insoluble material is present, the medium is microcentrifuged for 10 minutes at room temperature. The supernatant is transferred into a new tube. The pH is neutralized by adding 1.25 μl of 0.1 M Hepes per 10 μl.

2—Preparation of the Oligonucleotide Pairs

The oligonucleotide pairs are prepared as described in Example 1. Reported here are the results relating to the oligonucleotide pair D (SEQ ID No. 8 as 5′ oligonucleotide and SEQ ID No. 3 as 3′ oligonucleotide) according to the invention (cf. Table 1 below).

3—Amplification by the Polymerase Chain Reaction (PCR)

Amplification by the polymerase chain reaction is carried out as described in Example 1 by bringing the genomic DNA preparations obtained into contact with oligonucleotide pairs D which serve, in this case, as primers.

The products derived from the amplification are resolved on 2% agarose gel in parallel with molecular weight markers (M).

The results of electrophoretic resolution on 2% agarose gel of the PCR-derived products are illustrated by FIG. 2.

FIG. 2 illustrates the PCR amplification of the sequence encoding p50.2 from the genomic DNA of p50.2⁺ transgenic mouse splenocytes: illustrated therein is the result of the electrophoretic resolution of the products derived from PCR amplification after bringing the pair of primers D according to the invention (cf. Table 1) into contact with the DNA populations of splenocytes of p50.2⁺ transgenic mice (lanes+) or of p50.2⁻ non-transgenic mice (lanes−) The molecular weight markers are resolved in lane M.

The D primers according to the invention make it possible to recognize a fragment of 533 bp present on the DNA of p50.2⁺ murine splenocytes and absent from the DNA of p50.2⁻ murine splenocytes.

Similar results were obtained with the oligonucleotide pairs A, B and E to L presented in Table 1 below and also made it possible to document the desired receptors (cf. “molecule” column of Table 1).

The NKR and/or NKR counterpart repertoires thus documented can be, in particular with the aid of conventional biostatic studies, correlated with given physiological or pathological situations linked to these repertoires, and with the control of the activation of the cells expressing them in general.

TABLE 1

| | | | | | | Oligo 5′ |
|---|---|---|---|---|---|---|
| Oligo | | | | | Sequence of the oligo | sequence |
| pair | Molecule | Function | Tm | Name of oligo | (5′ --> 3′) | Oligo 3′ |
| A | p58.EB6 | Inhibitory | 56° C. | p58.1FOR | AGTCGCATGACGCAAGAC | Seq ID NO. 1 |
| | (p58.1) | | 54° C. | ITIM N-term BACK | CAACTGTG(T/C) (A/G)TATGTCAC | Seq ID NO. 5, 2, 6, 7 |
| B | p50.EB6 | Activatory | 56° C. | p58.1 FOR | AGTCGCATGACGCAAGAC | Seq ID NO. 1 |
| | (p50.1) | | 49° C. | TM-ACT BACK | GATGGTGAAAGGGATTTT | Seq ID NO. 3 |
| C | p58.183 | Inhibitory | 56° C. | p58.2 FOR | GGTCCCATGATGCAAGAC | Seq ID NO. 4 |
| | (p58.2) | | 54° C. | ITIM N-term BACK | CAACTGTG(T/C) (A/G)TATGTCAC | Seq ID NO. 5, 2, 6, 7. |
| D | p50.183 | Activatory | 56° C. | p58.2 FOR | GGTCCCATGATGCAAGAC | Seq ID NO. 8 |
| | (p50.2) | | 49° C. | TM-ACT BACK | GATGGTGAAAGGGATTTT | Seq ID NO. 3 |
| E | p70.INH | Inhibitory | 58° C. | p70.FRO | CCCGTGGTGATCATGGTC | Seq ID NO. 9 |
| | | | 54° C. | ITIM N-term BACK | CAACTGTG(T/C) (A/G)TATGTCAC | Seq ID NO. 5, 2, 6, 7. |
| F | p70.ACT | Activatory | 58° C. | p70.FOR | CCCGTGGTGATCATGGTC | Seq ID NO. 9 |
| | | | 49° C. | TM-ACT BACK | GATGGTGAAAGGGATTTT | Seq ID NO. 3 |
| G | p140.INH | Inhibitory | 56° C. | ITIM N-term.FOR | GTGAC(A/G)TAC(A/G)CACAGTTG | Seq ID NO. 10, 11, 12, 13 |
| | | | 58° C. | Ext C-term BACK | ACCTGACTGTGGTGCTCG | Seq ID NO. 14 |
| H | p140.ACT | Activatory | 58° C. | p140.FOR | ACCTACAGATGTTATGGTTCTGTT | Seq ID NO. 15 |
| | | | 49° C. | TM-ACT BACK | GATGGTGAAAGGATTTT | Seq ID NO. 13 |
| I | NKG2A | Inhibitory | 54° C. | NKG2A FOR | TCTACATTAATACAGAGGCAC | Seq ID NO. 16 |
| | | | 54° C. | NKG2A/B/C.BACK | ATCTATAGAAAGCAGACT | Seq ID NO. 17 |
| J | NKG2B | Inhibitory | 52° C. | NKG2B.FOR | ATTCCCTCACGTCATTGT | Seq ID NO. 18 |
| | | | 54° C. | NKG2A/B/C.BACK | ATCTATAGAAAGCAGACT | Seq ID NO. 17 |
| K | NKG2C | Activatory | 54° C. | NKG2C.FOR | AGTAAACAAAGAGGAACCTTC | Seq ID NO. 19 |
| | | | 54° C. | NKG2A/B/C.BACK | ATCTATAGAAAGCAGACT | Seq ID NO. 17 |
| L | NKG2D | Activatory | 56° C. | NKG2D.FOR | AGCAAAGAGGACCAGGATTTA | Seq ID NO. 20 |
| | | | 58° C. | NKG2D.BACK | CACAGTCCTTTGCATGCAGAT | Seq ID NO. 21 |
| M | CD56 | | 51° C. | 5′ hCD56 | ATCCAGTACACTGATGAC | Seq ID NO. 22 |
| | | | 54° C. | 3′ hCD56 | GTCGATGGATGGTGAAGA | Seq ID NO. 23 |
| N | Actine | | 62° C. | 5′ Actine | TACCACTGGCATCGTGATGGACT | Seq ID NO. 24 |
| | | | 64° C. | 3′ Actine | TCCTTCTGCATCCTGTCGGCAAT | Seq ID NO. 25 |

TABLE 2

| | | Genbank |
|---|---|---|
| Oligonucleotide | Name of the | accession |
| pair | cDNA | number |
| A | cl-42 | U24076 |
| | NKAT-I | L41267 |
| | cl-47.11 | U24078 |
| B | X98858 | X98585 |
| | X98892 | X98892 |
| | NKAT-7AA | L76670 |
| | NKAT-9AA | L76672 |
| C | cl-43 | U24075 |
| | NKAT-6AA | L76669 |
| | NKAT-2BA | L76663 |
| | cl-6 | U24074 |
| | NKAT-2 | L41268 |
| | KIR-023GB | U73395 |
| | NKAT-2AB | L76662 |
| | NKAT-3DA | L76664 |
| D | cl-49 | U24079 |
| | NKAT-5 | L41347 |
| | X89893 | X89893 |
| | cl-39 | U24077 |
| | NKAT-8 | L76671 |
| | NKAT-5DA | L76667 |
| E | X94262 | X94262 |
| | NKAT-3 | L41269 |
| | NKBI-1 | U31416 |
| | NKBI-2 | U33328 |
| | KIR-103AS | U71199 |
| | KIR-103AST | U73394 |
| | cl-1.1 | X94373 |
| | cl-11 | U30274 |
| | cl-2 | U30273 |
| F | NKAT-10 | L76661 |
| | KIR-123FM | U73396 |
| G | NKAT-4 | L41270 |
| | X94374 | X94374 |
| | X93595 | X93595 |
| | X93596 | X93596 |
| | NKAT-4BA | L76666 |
| | NKAT-4AA | L76665 |
| | cl-5 | U30272 |
| I | NKG2A | X54867 |
| J | NKG2B | X54868 |
| K | NKG2C | X54869 |
| L | NKG2D | X54870 |

1. In vitro method of documenting a repertoire of (an) NKR immunoreceptor(s) comprising in particular the KIR p58.1, p58.2, p70.INH and p140.INH, and the NKG2A and NKG2B receptors, and/or of a repertoire of (an) NKR immunoreceptor counterpart(s), comprising in particular the KAR p50.1, p50.2, p70.ACT and p140.ACT receptors, and the NKG2C, NKG2D, NKG2E and NKG2F receptors, these immunoreceptors being designated hereinafter target receptor(s), characterized in that it comprises:
i. the use of at least one pair of oligonucleotides, one being designated 3′ oligonucleotide and the other 5′ oligonucleotide, the 3′ and 5′ oligonucleotides of the same said pair both being capable, under hybridization conditions corresponding to incubation for 1 min in a buffer [20 mM Tris-HCl, pH 8.4; 50 mM KCl; 2.5 mM MgCl₂] at a temperature of between 50° C. and 65° C. approximately, of hybridizing to the DNA or to the cDNA of a target NKR receptor, or NKR counterpart, but not hybridizing, under the same hybridization conditions, with the DNA or the cDNA of an NKR receptor counterpart, or respectively of an NKR receptor, functional counterpart of the said target receptor,
ii. the bringing of DNA or cDNA populations of a biological sample of human or animal origin for which it is desired to document the repertoire of (a) target immunoreceptor(s), into contact with an excess of at least one 3′ and 5′ oligonucleotide pair according to i. under conditions favourable to the hybridization of this 3′ and 5′ oligonucleotide pair with the DNAs or cDNAs of the biological sample, and
iii. the detection of the possible hybrids formed between these DNAs or cDNAs and the 3′ and 5′ oligonucleotide pair(s).

2. Method according to claim 1, characterized in that the said or at least one of the said 3′ and 5′ oligonucleotide pair(s) used is in addition capable, undfder the same hybridization conditions as those defined under i., of not hybridizing to the DNA or CDNA of a receptor, either NKR or NKR counterpart, other than the said target receptor.

3. Method according to any one of the preceding claims, characterized in that the 5′ oligonucleotide of a said 3′ and 5′ oligonucleotide pair used for an NKR target receptor (or NKR counterpart) is capable, under the same said hybridization conditions, of hybridizing to the DNA or to the cDNA of an NKR receptor counterpart (or respectively NKR receptor), functional counterpart of the said NKR target receptor (or respectively NKR receptor counterpart).

4. Method according to any one of the preceding claims, characterized in that the 3′ oligonucleotide of a said 3′ and 5′ oligonucleotide pair, used for a KAR target receptor, is capable, under the same said hybridization conditions, of hybridizing to the DNA or cDNA of the said KAR target receptor at the level of a nucleotide stretch which comprises a sequence corresponding, according to the universal genetic code, and taking into account the degeneracy of the said code, to the amino acid sequence Lys Ile Pro Phe Thr Ile (K I P F T I) or Lys Leu Pro Phe Thr Ile (K L P F T I) (SEQ ID No. 26 or 27).

5. Method according to any one of the preceding claims, characterized in that the said (or at least one of the said) 3′ and 5′ oligonucleotide pair(s) having as target receptor a KIR receptor is chosen from the group of 3′ and 5′ oligonucleotide pairs consisting of:
a 5′ oligonucleotide comprising the sequence SEQ ID No. 1, or a sequence which is derived therefrom, and at least one 3′ oligonucleotide comprising the sequence SEQ ID No. 5, No. 2, No. 6 or No. 7, or a sequence which is derived therefrom,
a 5′ oligonucleotide comprising the sequence SEQ ID No. 4, or a sequence which is derived therefrom, and at least one 3′ oligonucleotide comprising the sequence SEQ ID No. 5, No. 2, No. 6 or No. 7, or a sequence which is derived therefrom,
a 5′ oligonucleotide comprising the sequence SEQ ID No. 9, or a sequence which is derived therefrom, and at least one 3′ oligonucleotide comprising the sequence SEQ ID No. 5, No. 2, No. 6 or No. 7, or a sequence which is derived therefrom,
at least one 5′ oligonucleotide comprising the sequence SEQ ID No. 10, No. 11, No. 12 or No. 13, or a sequence which is derived therefrom, and a 3′ oligonucleotide comprising the sequence SEQ ID No. 14, or a sequence which is derived therefrom.

6. Method according to any one of the preceding claims, characterized in that the said (or at least one of the said) 3′ and 5′ oligonucleotide pair(s) having as target receptor a KAR receptor is chosen from the group of 3′ and 5′ oligonucleotide pairs consisting of:
a 5′ oligonucleotide comprising the sequence SEQ ID No. 1, or a sequence which is derived therefrom, and a 3′ oligonucleotide comprising the sequence SEQ ID No. 3, or a sequence which is derived therefrom,
a 5′ oligonucleotide comprising the sequence SEQ ID No. 8, or a sequence which is derived therefrom, and a 3′ oligonucleotide comprising the sequence SEQ ID No. 3, or a sequence which is derived therefrom,
a 5′ oligonucleotide comprising the sequence SEQ ID No. 9, or a sequence which is derived therefrom, and a 3′ oligonucleotide comprising the sequence SEQ ID No. 3, or a sequence which is derived therefrom,
a 5′ oligonucleotide comprising the sequence SEQ ID No. 15, or a sequence which is derived therefrom, and a 3′ oligonucleotide comprising the sequence SEQ ID No. 3, or a sequence which is derived therefrom.

7. Method according to any one of the preceding claims, characterized in that the said (or at least one of the said) 3′ and 5′ oligonucleotide pair(s) having as target receptor a NKG2 receptor is chosen from the group of 3′ and 5′ oligonucleotide pairs consisting of:
a 5′ oligonucleotide comprising the sequence SEQ ID No. 16, or a sequence which is derived therefrom, and a 3′ oligonucleotide comprising the sequence SEQ ID No. 17, or a sequence which is derived therefrom,
a 5′ oligonucleotide comprising the sequence SEQ ID No. 18, or a sequence which is derived therefrom, and a 3′ oligonucleotide comprising the sequence SEQ ID No. 17, or a sequence which is derived therefrom,
a 5′ oligonucleotide comprising the sequence SEQ ID No. 19, or a sequence which is derived therefrom, and a 3′ oligonucleotide comprising the sequence SEQ ID No. 17, or a sequence which is derived therefrom,
a 5′ oligonucleotide comprising the sequence SEQ ID No. 20, or a sequence which is derived therefrom, and a 3′ oligonucleotide comprising the sequence SEQ ID No. 21, or a sequence which is derived therefrom.

8. Method according to any one of the preceding claims, characterized in that the two 3′ or 5′ oligonucleotides of the same said pair are each coupled to a marker, in particular coupled to a fluorescent or radioactive marker, such as ³²p, allowing the visualization of the hybrids which they may form with the said DNA or cDNA populations of the said biological sample.

9. Method according to any one of the preceding claims, characterized in that the said 3′ and 5′ oligonucleotide pair(s) serve(s) as 3′ and 5′ primers, respectively, for extension by DNA polymerase.

10. Method according to any one of the preceding claims, characterized in that the said hybrids which may be formed are amplified by at least one PCR prior to their detection.

11. Method according to any one of the preceding claims, characterized in that the said hybrids which may be formed are amplified by nested PCR.

12. Method according to any one of the preceding claims, characterized in that the said detection of the hybrids which may be formed comprises, in addition, the resolution, on a polyacrylamide gel, of the reaction mixture derived from the bringing into contact, as well as the visualization of the presence or of the absence of electrophoretic bands containing the said hybrids which may be formed.

13. Method according to any one of the preceding claims, characterized in that it is applied to the documentation of a genotypic repertoire of NKR immunoreceptors and/or of NKR immunoreceptor counterparts.

14. Method according to any one of the preceding claims, characterized in that it is applied to the documentation of an expression repertoire of NKR immunoreceptors and/or of NKR immunoreceptor counterparts.

15. Method according to any one of the preceding claims, characterized in that the said biological sample of human or animal origin is peripheral blood, bone marrow, lymphocytes, NK and/or T cells, transgenic cells expressing immunoreceptors and a fraction isolated from these samples.

16. Method according to any one of the preceding claims, characterized in that it is applied to the screening of a library of organs, tissues or cells.

17. Method according to any one of the preceding claims, characterized in that it is applied to the prediction or to the monitoring of the acceptance or rejection, by a human or an animal, of cells, tissue or organ which is (are) genetically different.

18. Method according to any one of the preceding claims, characterized in that it is applied to the prediction or to the monitoring of the safety or of the pathogenicity (GVH), for a human or an animal, of a graft or transplant, of cells, tissue or organ which is (are) genetically different.

19. Method according to any one of the preceding claims, characterized in that it is applied to the prediction or to the monitoring, for a human or an animal, of a GVL-type effect on the part of cells, tissue or organ which is (are) genetically different.

20. Method according to any one of the preceding claims, characterized in that it is applied to the determination of the state of activation of NK and/or T cells at a given instant in an Animal or a human.

21. Method according to any one of the preceding claims, characterized in that it is applied to the prediction or to the monitoring of the state of resistance of an animal or a human towards a viral infection, such as an HIV infection, or a parasitic infection, such as malaria, or a bacterial infection, towards an autoimmune disease, such as rheumatoid arthritis, or alternatively towards the development of malignant cells such as leukaemia cells.

22. Method according to any one of the preceding claims, characterized in that it is applied to the screening of medicaments which are active on infectious diseases, on autoimmune diseases and on tumour diseases.

23. Kit for carrying out the method according to any one of claims 1 to 22, characterized in that it comprises, in a container, at least one said 3′ and 5′ oligonucleotide pair, the reagents for carrying out the said method(s) such as a buffer, a marker (optionally coupled to the oligonucleotides of the said pair), as well as instructions for use.
